(19) United States
(12) Patent Application Publication
(10) Pub. No.: US 2010/0137398 A1
(43) Pub. Date: Jun. 3, 2010

(54) USE OF HDAC INHIBITORS FOR THE TREATMENT OF GASTROINTESTINAL CANCERS

(75) Inventor: Peter Wisdom Atadja, Acton, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/594,613

(22) PCT Filed: May 2, 2008
(86) PCT No.: PCT/US08/62341

§ 371 Date: Oct. 5, 2009

Related U.S. Application Data
(60) Provisional application No. 60/915,966, filed on May 4, 2007.

(51) Int. Cl. A61K 31/404; A61P 35/00

(52) U.S. Cl. 514/415

(57) ABSTRACT

The present invention relates to the use of an HDAC inhibitor for the preparation of a medicament for the treatment of gastrointestinal cancers; a method of treating a warm-blooded animal, especially a human, having gastrointestinal cancer, comprising administering to said animal a therapeutically effective amount of an HDAC inhibitor, especially a compound of formula (I) as defined herein; and to a pharmaceutical composition and a commercial package.

### FIELD OF THE INVENTION

The present invention relates to the use of an HDAC inhibitor for the preparation of a medicament for the treatment of gastrointestinal cancers; a method of treating a warm-blooded animal, especially a human, having gastrointestinal cancer; comprising administering to said animal a therapeutically effective amount of an HDAC inhibitor, especially a compound of formula (I), as defined herein; and to a pharmaceutical composition and a commercial package.

### BACKGROUND OF THE INVENTION

Patients suffering from gastrointestinal cancers have low overall survival rates. The standard treatment of chemotherapy, is not always effective. Therefore, there is a need to develop novel treatment methods.

### SUMMARY OF THE INVENTION

The term “gastrointestinal cancers”, as used herein, includes, but is not limited to, hepatocellular carcinoma and/or pancreatic cancer.

The compounds of formula (I), as defined herein, are histone deacetylase inhibitors (HDAC inhibitors); Reversible acetylation of histones is a major regulator of gene expression that acts by altering accessibility of transcription factors to DNA. In normal cells, histone deacetylase (HDA) and histone acetyltrasferase together control the level of acetylation of histones to maintain a balance. Inhibition of HDA results in the accumulation of hyperacetylated histones, which results in a variety of cellular responses.

Surprisingly, it was now found that HDAC inhibitors, especially the compounds of formula (I), as defined herein, directly inhibit the proliferation of gastrointestinal cancer, such hepatocellular carcinoma and/or pancreatic cancer.

Hence, the invention relates to the use of an HDAC inhibitor for the preparation of a medicament for the treatment of gastrointestinal cancer.

### DETAILED DESCRIPTION OF THE DRAWINGS

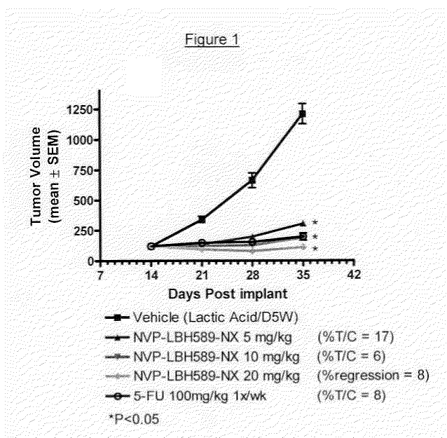

FIG. 1 illustrates that LBH589 treatment inhibits tumor growth in the HCT116 xenograft model.

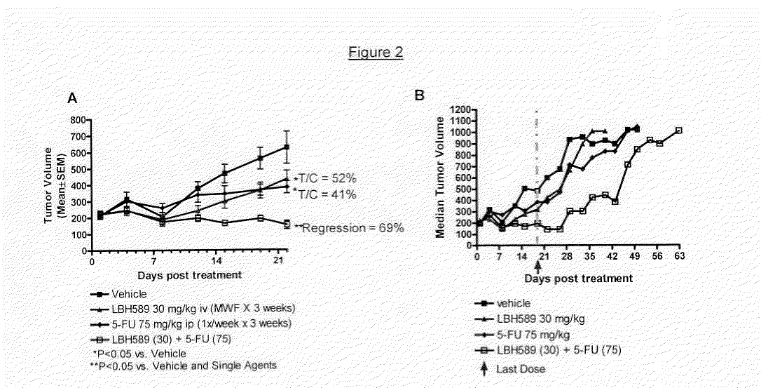

FIG. 2 illustrates co-treatment of LBH589 with 5-Fluorouracil enhances tumor growth inhibition and tumor growth delay in Colo205 colon cancer xenograft model.

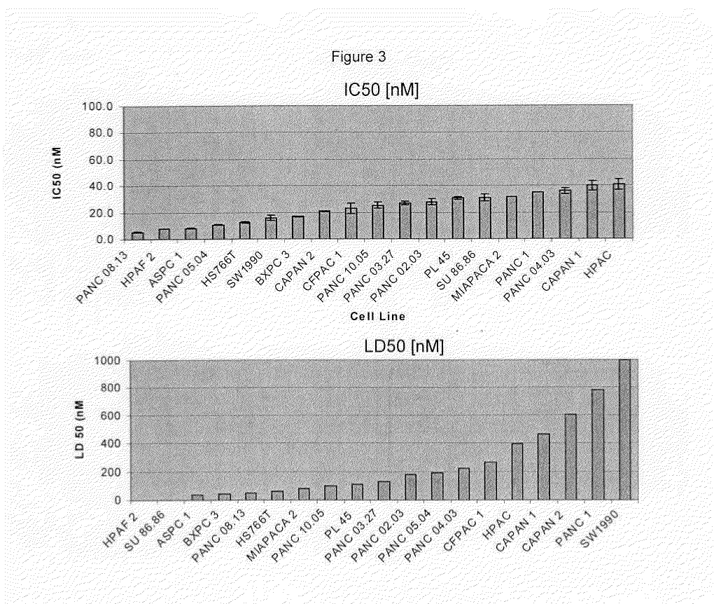

FIG. 3 illustrates the anti-proliferative and cytotoxic effects of LBH589 in 19 pancreatic cancer cell lines.

### DETAILED DESCRIPTION OF THE INVENTION

HDAC Inhibitor Compounds

HDAC inhibitor compounds of particular interest for use in the inventive combination are hydroxamate compounds described by the formula (I):

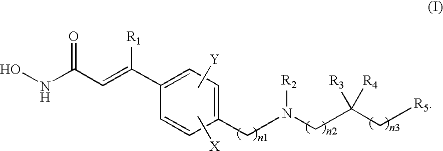

wherein

R₁ is H; halo; or a straight-chain C₁-C₈alkyl, especially methyl, ethyl or n-propyl, which methyl, ethyl and n-propyl substituents are unsubstituted or substituted by one or more substituents described below for alkyl substituents;

R₂ is selected from H; C₁-C₁₀alkyl, preferably C₁-C₈alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; (CH₂)ₙOC(O)R₆; amino acyl; HON—C(O)—CH═C(R₁)-aryl-alkyl-; and —(CH₂)ₙR₇;

R₃ and R₄ are the same or different and independently H, C₁-C₆alkyl, acyl or acylamino, or R₃ and R₄, together with the carbon to which they are bound, represent C═O, C═S or C═NR₈, or

R₂, together with the. nitrogen to which it is bound, and R₃, together with the carbon to which it is bound, can form a C₄₋C₉heterocyeloalkyl, a heteroaryl, a polyheteroaryl , a non-aromatic polyheterocycle, or a mixed aryl and non-aryl polyheterocycle ring;

R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;

n, n₁, n₂ and n₃ are the same or different and independently selected from 0-6, when n₁ is 1-6, each carbon atom can be optionally and independently substituted with R₃ and/or R₄;

X and Y are the same or different and independently selected from H; halo; C₁-C₄alkyl, such as CH₃ and CF₃; NO₂; C(O)R₁; OR₉; SR₉; CN; and NR₁₀R₁₁;

R₆ is selected from H; C₁-C₈alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;

R₇ is selected from OR₁₅, SR₅, S(O)R₁₆, SO₂R₁₇, NR₁₃R₁₄ and NR₁₂SO₂R₆;

R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;,

R₉ is selected from C₁-C₄alkyl, e.g., CH₃ and CF₃; C(O)-alkyl, e.g., C(O)CH₃; and C(O)CF₃;

R₁₀ and R₁₁ are the same or different and independently selected from H, C₁-C₄alkyl and —C(O)-alkyl;

R₁₂ is selected from H; C₁-C₆alkyl; C₄-C₉cycloalkyl; C₄C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;

R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or

R₁₃ and R₁₄, together with the nitrogen to which they are bound, are C₄-C₉heterocycloalkyl, heteroaryl, polyheteroaryl, non-aromatic polyheterocycle or mixed aryl and non-aryl polyheterocycle;

R₁₅ is selected from H, C₁-C₆alkyl, C₄-C₉cycloalkyl, C₄-C₉heterocyeloalkyl, aryl, heteroaryl, arylalkyl, heteroarylalkyl and (CH₂)ₘZR₁₂;

R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;

R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;

m is ah integer selected from 0-6; and

Z is selected from O; NR₁₃; S; and S(O), or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₈alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents. include 3- to 9-membered aliphatic rings, such as 4- to 7-membered aliphatic-rings, containing from 1-3heteroatoms selected from nitrogen, sulfur, oxygen. Examples of Suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉Cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include Compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; hitro; amino; alkylamino; aminoalkyl; alkyl ketones; nitrite; carboxyalkyl; alkylsulfonyl; aminosulfonyl; arylsulfonyl and OR₁₅, such as alkoxy. Preferred, substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazdlyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁—(CH-aryl)—(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like;

Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅₋₁heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ-C(H)(NR₁₃R₁₄)—(CH₂)ₙ-R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±-3-amin-4-hexenoyl.

Non-aromatic polycycle substituents include bicyclic and tricyclic fused ring systems where each ring can be 4- to 9-membered and each ring can contain zero, one or more double and/or triple bonds. Suitable examples of non-aromatic polycycles include decalin, octahydroindene, penthydrobenzocycloheptene and perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, e.g., 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridinel indole, benzofuran, benzothlofuran, benzindole, benzoxazble, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂)₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic: Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane-sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula:

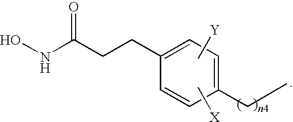

Preferences for each of the substituents include the following:
R₁ is H, halo or a straight-chain C₁-C₄alkyl;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl, or
R₃ and R₄, together with the carbon to which they are bound; represent C═O, C═S or C═NR₈;
R₅ is selected from H, C₁C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n, is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, ON and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄₋C₉cycloalkyl, C₄-C₉heterocycloalkyl; aryl, heteroaryl, arylalkyl, heteroarylalkyl and NR₁₃R₁₄;
m is an integer selected from 0-6; and
Z is selected from O, NR₁₃, S and S(O); or a pharmaceutically acceptable salt thereof.

Useful compounds of the formula (I), include those wherein each of R₁, X, Y, R₃ and R₄ is H, including those wherein one of n₂ and n₃ is 0and the other is 1, especially those wherein R₂ is H or —CH₂—CH₂—OH.

One suitable genus of hydroxamate compounds are those of formula (Ia):

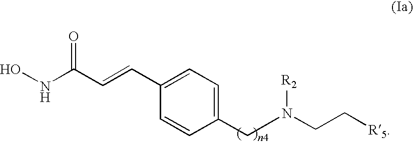

wherein

n₄ is 0-3;

R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and

R₅ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;

or a pharmaceutically acceptable salt thereof.

Another suitable genus of hydroxamate compounds are those of formula (Ia):

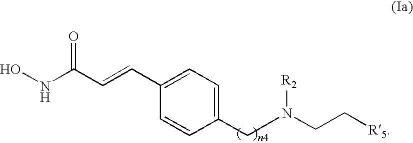

wherein

n₄ is 0-3;

R₂is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, aminoacyl and —(CH₂)ₙR₇;

R₅ is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, arid p-C₁-C₄ alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl; or a pharmaceutically acceptable salt thereof.

Another interesting genus is the compounds of formula (Ib):

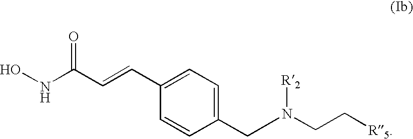

wherein

R₂ is selected from H, C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and

R₅ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;

or a pharmaceutically acceptable salt thereof.

Another interesting genus of hydroxamate compounds are the compounds of formula (Ic):

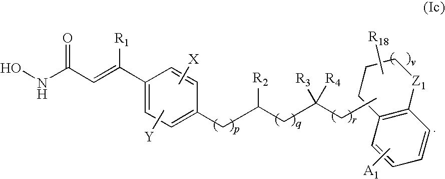

wherein

the ring containing Z₁ is aromatic non-aromatic, which non-aromatic rings are saturated or unsaturated,

Z, is O, S or N-R₂₀;

R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;

R₂₀ is H; C₁-C₆alkyl; C₁-C₆alkyl-C₃-C₉cloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl and toluenesulfonyl;

A₁ is 1, 2 or 3 substituents which are independently H; C₁-C₆alkyl; —OR₁₉; halo; alkylamino; aminoalkyl; halo; or heteroarylalkyl, e.g., pyridylmethyl;

R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl and —(CH₂-CH═CH(CH₃)(CH₂))₁₋₃H;

R₂ is selected from H, C₁-C₆alkyl, C₄-C₉cycloalkyl, C₄-C₉heterocycloalkyl, cycloalkylalkyl, aryl, heteroaryl, arylalkyl; heteroarylalkyl, —(CH₂)ₙC(O)R₆, amino acyl and —(CH₂)ₙR₇;

v is 0, 1 or 2;

p is 0-3; and

q is 1-5 and r is 0; or

q is 0 and r is 1-5;

or a pharmaceutically acceptable salt thereof. The other variable substituents are as defined above;

Especially useful compounds of formula (Ic), are those wherein R₂ is H, or —(CH₂)ₚCH₂OH, wherein p is 1-3, especially those wherein R₂ is H; such as those wherein R₁ is H and X and Y are each H, and wherein q is 1-3 and r is 0 or wherein q is 0 and r is 1-3, especially those wherein Z₁ is N-R₂₀. Among these compounds R₂, is preferably H or —CH₂-CH₂OH and the sum of q and r is preferably 1.

Another interesting genus of hydroxamate compounds are the compounds of formula (Id):

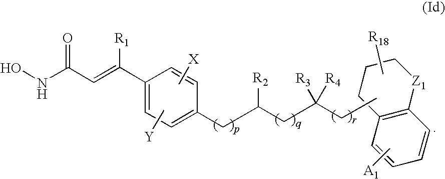

wherein

Z, is O, S or N-R₂₀;

R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl;

R₂₀ is H; C₁-C₆alkyl, C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);

A₁ is 1, 2 or 3 substituents which are independently H, C₁-C₆alkyl, —OR,₁₉ or halo;

R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;

p is 0-3; and

q is 1-5 and r is 0; or

q is 0 and r is 1-5;

or a pharmaceutically acceptable salt thereof. The other variable substituents are as defined above.

Especially useful compounds of formula (Id), are those wherein R₂ is H or —(CH₂)ₚCH2OH, wherein p is 1-3, especially those wherein R₂ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie):

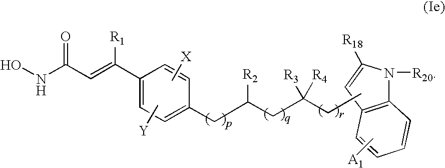

or a pharmaceutically acceptable salt thereof. The variable substituents are as defined above.

Especially useful compounds of formula (Ie), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (Ie), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is Hand X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further relates to the compounds of the formula (If):

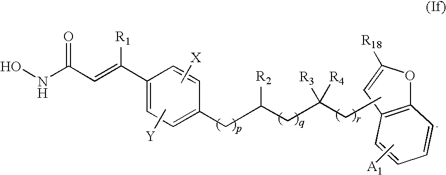

or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H; such as those wherein R₁ is H and X and Y are each H, and wherein q is 1 -3 and r is 0 or wherein q is 0 and r is 1-3. Among these compounds R₂ is preferably H or —CH2—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, e.g., metal salts, such as alkali and alkaline earth metal salts, ammonium salts, organic amine addition salts and amino acid addition salts and sulfonate salts. Acid addition salts include inorganic acid addition salts; such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577. Two preferred compounds within the scope of WO 02/22577 are:

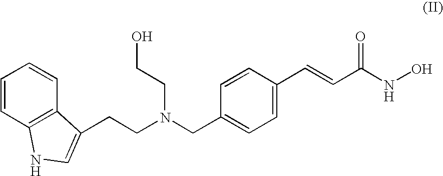

N-hydroxy-3-[4-[(2-hydroxyethyl)(2-(1H-indol-3-yl)ethyl]-amino]methyl]phenyl]-2E-2-propenamide, or a pharmaceutically acceptable salt thereof; and

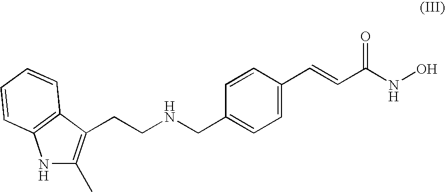

N-hydroxy-3-[4-[[[2-(2-methyl-1H-indol-3-yl)ethyl]-amino]methyl]phenyl]-2E-2-propenamide, or a pharmaceutically acceptable salt thereof.

The present invention pertains in particular to the use of HDAC inhibitors for the preparation of a medicament for the treatment of gastrointestinal cancer.

An HDAC inhibitor as used for the present invention displays in the assay described above preferably an IC₅₀ value between 50 and 2500 nM, more preferably, between 250 and 2000 nM, and most preferably between 500 and 1250 nM.

Furthermore, the invention relates to a method of treating gastrointestinal cancer, especially hepatocellular carcinoma or pancreatic cancer, comprising administering a therapeutically effective amount of an HDAC inhibitor to a warm-blooded animal, in particular a human, in need thereof, preferably a therapeutically effective amount of a compound of formula (I), as defined above, or the salt of such compound having at least one salt-forming group; to a warm-blooded animal, preferably a human, in need thereof.

The term “treatment”, as used herein, comprises the treatment of patients having gastrointestinal cancer or being in a pre-stage of said cancer which effects the delay of progression of the disease in said patients.

The present invention provides a method of treating gastrointestinal cancer, especially hepatocellular carcinoma or pancreatic cancer, comprising administering an HDAC inhibitor in an amount which is therapeutically effective against gastrointestinal cancer, especially hepatocellular carcinoma or pancreatic cancer to a warm-blooded animal in need thereof.

The person skilled in the pertinent art is fully enabled to select relevant test models to prove the hereinbefore and hereinafter mentioned beneficial effects on gastrointestinal cancer, of a compound inhibiting the HDAC activity. The pharmacological activity of a compound inhibiting the HDAC activity may, e.g., be demonstrated in a suitable clinical study or by means of the Examples described below.

The present invention also provides the. use of a compound of formula (I), as defined herein, and the use of a COMBINATION OF THE INVENTION for the preparation of a medicament for the treatment of lymphoproliferative diseases.

### EXAMPLES

### Example 1

### Monolayer Growth Inhibition Assay

The MTT is a colorimetric assay to determine the cell proliferation rate. The yellow tetrazolium MTT (3-(4, 5-dimethylthiazolyl-2)-2, 5-diphenyltetrazolium bromide) is reduced, by metabolically active cells, in part by the action of dehydrogenase enzymes, to generate reducing equivalents such as NADH and NADPH. The resulting intracellular purple formazan can be solubilized and quantified by spectrophotometric means. The signals produced is directly proportional to the cell numbers. Describing the MTT assay in detail, experiments were done using six-point or 9 point drug titrations in multi-well tissue culture dishes, with outer rows left empty. Cells were suspended in complete media at densities of between 10³ and 10⁴ cell/ml, respectively, and added per well. The appropriate medium (200 μl) was then added. Twenty-four hours later, 10 μl of MTS solution, were added to one plates to determine the activity at the time of compound addition (T₀). This plate was incubated at 37° for 4 hours and the optical density was measured on a Molecular Devices Thermomax at 490 nm using the Softmax program. The T₀ plate served as a reference for initial activity at the beginning of the experiment.

Compound addition began 24 hours after seeding, the same time as the T₀ determination. Serial dilutions at 4-fold 2-fold, 1-fold, 0.5-fold, 0.25-fold and 0.125-fold of previously determined IC₅₀ values of each compound were made in a 96-deep well plate with the highest concentrations on the edge of the plate. Each of the six dilutions were added in triplicate and complete medium was added to the empty outer rows, without cells. The compounds were added to the plates singly or in combination with Compound III (LBH589). The plates were incubated at 37° C. for 72 hours from seeding. The MTS solution was added (as for the T₀ plate) and read four hours later. In order to analyze the data, the average value of media alone (background) was subtracted from each experimental well and the triplicate values were averaged for each compound dilution. The following formulas were used to calculate percent growth.

If X>T₀, % Growth=100×(X−T₀)/(GC−T₀))

If X<T₀, % Growth=100×(X−T₀)/T₀)

T₀=average value of T₀ minus background

GC=average value of untreated cells (in triplicate) minus background
X=average value, of compound treated cells (in triplicate) minus background

IC50 the concentration of LBH589 required to inhibit cell growth by 50% and LD50s the concentration required to reduce cell number (kill cells) to 50% the original innoculum were determined. The “% Growth” was plotted against compound concentration and used to calculate IC50s and LD50s, employing the user-defined spline function in Microsoft Excel.

The Anti-proliferation and cytotoxic effects of LBH589 in a large panel of 36 colon cancer cell lines are described in the attached Table:

NVP-LBH589

| Cell Line | IC50 (nM) | LD50 (nM) |
|---|---|---|
| SW620 | 0.79 | 1.70 |
| SW480 | 1.48 | 6.90 |
| SW403 | 2.03 | 9.84 |
| SW837 | 0.66 | 11.46 |
| SW48 | 1.29 | 13.09 |
| KM20L2 | 14.62 | 16.95 |
| Colo201 | 15.12 | 20.14 |
| CL-11 | 3.54 | 25.92 |
| SNU-C1 | 4.51 | 26.07 |
| SW1116 | 2.28 | 26.93 |
| HT115 | 3.49 | 31.02 |
| T84 | 2.16 | 32.82 |
| Colo205 | 14.68 | 33.52 |
| Colo741 | 9.22 | 33.73 |
| SW948 | 5.06 | 34.92 |
| WiDr | 4.39 | 41.86 |
| Colo678 | 5.60 | 45.52 |
| CaCo2 | 13.84 | 45.55 |
| MDST8 | 4.87 | 48.44 |
| HCT116 | 7.11 | 51.72 |
| LOVO | 4.37 | 52.01 |
| HT29 | 2.83 | 70.23 |
| HCT8 | 4.94 | 77.70 |
| Colo320 | 6.50 | 82.90 |
| LS174T | 16.77 | 87.41 |
| HCC2998 | 29.67 | 154.87 |
| LS1034 | 76.70 | 227.83 |
| C2Bbe-1 | 27.46 | 243.12 |
| DLD-1 | 61.40 | 296.27 |
| HT55 | 8.21 | 334.52 |
| C170 | 62.29 | 382.03 |
| Colo320DM | 29.48 | 1103.37 |
| RKO | 5.57 | 2346.13 |
| Colo206F | 10.13 | >10000 |
| Colo320HSR | 13.82 | >10000 |
| HCT116 Bax −/− | 1.73 | >10000 |

Colon cancer cell lines were treated with DMSO vehicle control or varying concentrations of LBH589 for 3 days. Cell proliferation was measured on the day of cell plating and on the third day post-treatment. IC50 and LD50 values were calculated as described above. LBH589 exhibits potent antiproliferative effect on all 36 colon cancer cell lines examined, as demonstrated by the low nanomolar concentrations of IC50 values. LBH589 also exhibits potent cytotoxic effect in the great majority of the colon cancer cell lines tested with LD50<1 μM (n=31).

### Example 2

Female athymic nude mice were implanted subcutaneously with HCT116 colon cancer cells. When tumors reached a medan tumor volume of 120 mm³, mice were randomized into groups of 8 mice. Mice were treated with LBH589 at 5, 10 or 20 mg/kg intravenously (iv) 5 times a week for 3 weeks or 75 mg/kg of 5-Fluorouracil intravenously once a week for 3 weeks. Animals were calipered weekly. Compound activity was determined as the percent change in tumor volume of treated animals over control animals (% T/C). The percentage of regression was determined as the percent change in the final tumor volume at the end of the study over the starting tumor volume. Treatment with LBH589 at 5 or 10 mg/kg inhibited HCT116 tumor growth with % T/C of 17% and 6% respectively. Treatment with LBH589 at 20 mg/kg resulted in tumor regression of 8%. The results are described in FIG. 1.

### Example 3

Female athymic nude mice-were implanted subcutaneously with Col6205 colon cancer cells. When tumors reached a medan tumor volume of 220 mm3, mice were randomized into groups of 10 mice. Mice were treated with LBH589 at 30 mg/kg intravenously on Monday, Wednesday, Friday per week for 3 weeks, 75 mg/kg of 5-Fluorouracil intraperitoneally once a week for 3 weeks, or combination of the two agents. For tumor growth inhibition (FIG. 2A), compound activity was measured as the percent change in tumor volume of treated animals over control animals (% T/C). The percentage of regression was determined as the percent change in the final tumor volume at the end of the study over the starting tumor volume. For tumor growth delay (FIG. 2B), compound activity was measured as the change in median calculated time to the study end point (TTE), which was predefined as tumor volume of 1000 mm³. Median TTE for vehicle, LBH, 5-FU, and combined treatment was 39.5 days, 32.9 days, 44.5 days, arid 57.1 days respectively. Treatment with LBH589 or 5-FU single agent led to tumor growth inhibition (TGI) and tumor growth delay (TGD). More importantly, the combined treatment significantly enhanced both TGI and TGD. FIG. 2 illustrates the results.

### Example 4

Pancreatic cancer cell lines were treated with DMSO vehicle control or varying concentrations of LBH589 for 3 days. Cell proliferation was measured on the day of cell plating and on the third day post-treatment. IC50 and LD50 values were calculated as described above. LBH589 exhibits potent anti-proliferative effect on all 12 pancreatic cancer cell lines examined, as demonstrated by the low nanomolar concentrations of IC50 values. LBH589 also exhibits strong cytotoxic effect in the majority of the pancreatic cancer cell lines tested with LD50 <1 μM (n=10).

Table 2 describes the anti-proliferative and cytotoxic effects of LBH589 in a panel of 12 pancreatic cancer cell lines.

NVP-LBH589

| Cell Lines | IC50 [nM] | LD50 [nM] |
|---|---|---|
| MiaPaCa2 | 14.1 | 59.5 |
| BxPC3 | 15.9 | 104.8 |
| Hs766T | 14.1 | 119.9 |
| SU.86.86 | 51 | 170.4 |
| Panc1 | 18.9 | 175.4 |
| Capan1 | 55.6 | 191.5 |
| Panc3.27 | 38.5 | 276.8 |
| Panc10.05 | 31.8 | 282.4 |
| AsPC1 | 25.1 | 372.4 |
| Panc2.03 | 22.4 | 599.5 |
| HPAC | 122.7 | 2051.6 |
| Panc4.03 | 20.3 | >10000 |

A panel of 19 pancreatic cancer cell lines was independently assessed in cell proliferation assays. Cells were treated with DMSO vehicle control or varying concentrations of LBH589 for 6 days. Consistent with results presented in Table 2, LBH589 exhibits potent anti-proliferative effect on all 19 pancreatic cancer cell lines, showing low nanomolar concentrations of IC50 values. LBH589 also exhibits potent cytotoxic effect in 18 of the 19 pancreatic cancer cell lines, with LD50<1 μM. The results are described in FIG. 3.

1-5. (canceled)

6. A method of treating gastrointestinal cancer comprising administering a therapeutically effective amount of an HDAC inhibitor to a warm-blooded animal in need thereof:

7. A method according to claim 6, comprising administering a therapeutically effective amount of a compound of formula (I): 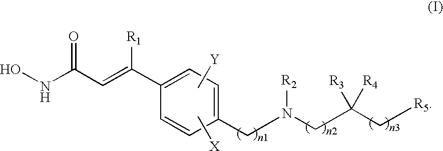
wherein
R₁ is H; halo; or a straight-chain C₁-C₆alkyl, especially methyl, ethyl or n-propyl, Which methyl, ethyl and n-propyl substituents are unsubstituted or substituted one or more substituents described below for alkyl substituents;
R₂ is selected from H; C₁-C₁₀alkyl, preferably C₁-C₆alkyl, e.g., methyl, ethyl or —CH₂—CH₂—CH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and (CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄₋C₉cycloalkyl; C₄-C₉heterocycloalkyl; acyl; aryl; heteroaryl; arylalkyl, e.g., benzyl; heteroarylalkyl e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6; when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycoalkyl; C₄-C₉heterocycloalkyl; cycloalkylalkyl, e.g., cyclopropylmethyl; aryl; heteroaryl; arylalkyl, e.g., benzyl and 2-phehylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄- C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from O; NR₁₃; S; and S(O), or a pharmaceutically acceptable salt thereof to a warm-blooded animal in need thereof.

8. The method according to claim 6, wherein the compound of formula (I) is N-hydroxy-3-[4-[[[2-(2-methyl-1H-indol-3-yl)-ethyl]-amino]methyl]phenyl]-2E-2-propenamide having the formula (III): 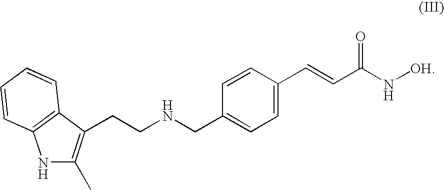
or a pharmaceutically acceptable salt thereof.

9. The method according to claim 6, wherein the gastrointestinal cancer is hepatocellular carcinoma or pancreatic cancer.

10. The method according to claim 6, wherein the warm-blooded animal is a human.
